(19) United States
(12) Patent Application Publication
(10) Pub. No.: US 2022/0252486 A1
(43) Pub. Date: Aug. 11, 2022

(54) METHOD AND APPARATUS FOR COLLECTING ODOR

(71) Applicant: TAESUNG ENVIRONMENTAL RESEARCH INSTITUTE CO., LTD., Ulsan (KR)

(72) Inventors: Gi Yeol YUN, Ulsan (KR); Seok Man KIM, Ulsan (KR)

(21) Appl. No.: 17/617,499

(22) PCT Filed: Dec. 10, 2020
(86) PCT No.: PCT/KR2020/018076

§ 371 Date: Dec. 8, 2021

(30) Foreign Application Priority Data
Dec. 24, 2019 (KR) 10-2019-0174043

(51) Int. Cl. G01N 1/24; B01D 53/30

(52) U.S. Cl. CPC G01N 1/24; B01D 53/30

(57) ABSTRACT

According to one embodiment, provided is a method for collecting odor, comprising: a step for acquiring odor monitoring information sensed by a plurality of odor sensors; a step for starting to collect odor in an odor collection bag on the basis of the odor monitoring information; a step for acquiring a limit message when the remaining capacity of the odor collection bag is less than or equal to a preset value as the odor is collected; and a step for outputting a notification indicating that replacement of the odor collection bag is required on the basis of the limit message.

### TECHNICAL FIELD

The present invention relates to a method and apparatus for collecting odor. More specifically, the present invention relates to a method and apparatus of notifying that replacement of an odor collection bag is required on the basis of the remaining capacity of the odor collection bag when collecting odor, thereby supporting a manager to easily replace the odor collection bag in a situation where it is difficult to predict when odor is generated, and preventing a situation where the odor collection bag is not enough because of frequent generation of unexpected odor and thus the odor cannot be collected.

### BACKGROUND ART

As the industry develops, the influence of odor generated from industrial complexes on the surrounding areas is becoming a social issue. Accordingly, the government enacted the odor prevention act and has legally controlled the amount of generated odor since 2005.

The diffusion degree of odor generated from pollution sources is determined by a terrain or an atmospheric condition, etc. When odor is generated from a specific point, in order to accurately track odor generating sources which affect the odor generation, it is necessary to obtain accurate information on atmospheric conditions, etc., at the time of odor generation. The atmospheric conditions can be measured if enough atmospheric measuring networks are set up. In addition, in order to backtrack the odor generating sources, it is necessary to obtain information on main pollutants produced from the odor generating sources, most of which has been secured by inspecting the process of the odor generating sources, etc.

In this situation, the most important information for the backtracking of the odor generating sources is component analysis of pollutants included when the odor is generated. For accurate component analysis, it is necessary to collect the gas at the time of odor generation in real time.

However, now, odor handling employees irregularly visit the area where civil complaints about odor generation often arise, carrying a simple portable device for collecting the air to collect the air manually. Odor tends to instantaneously appear and disappear due to atmospheric conditions, etc., which makes it difficult to collect the gas for accurate analysis.

Furthermore, the degree of sensing odor varies depending on individual's sense of smell, and the diffusion degree of odor is affected by atmospheric conditions, etc. Thus, for effective analysis, it is essential to accurately measure the concentration of odor and collect in real time the gas at the time of odor generation at the site upon odor generation.

However, the gas collecting at the site at the moment of initial stage of odor management depends on humans. That is, since odor managers visit the site and collect the gas on their own, they fail to collect the gas at the exact time of odor generation due to space/time constraints, resulting in inaccurate odor analysis, etc. As such, there are many problems in odor management.

### SUMMARY OF INVENTION

### Technical Task

An embodiment of the present invention provides a method and apparatus for collecting odor in order to solve the problems of the prior arts. More specifically, the present invention provides a method and apparatus of notifying that replacement of an odor collection bag is required on the basis of the remaining capacity of the odor collection bag when collecting odor, thereby supporting a manager to easily replace the odor collection bag in a situation where it is difficult to predict when odor is generated, and preventing a situation where the odor collection bag is not enough because of frequent generation of unexpected odor and thus the odor cannot be collected.

Also, the present invention records odor monitoring information and weather information on labels when collecting odor, thereby supporting the manager to efficiently manage the odor collection bag and odor information included therein.

The objects of the present disclosure are not limited to the aforementioned objects, and other objects that are not mentioned can be clearly understood from the following description.

### Means for Solving the Task

The method for collecting odor according to a first aspect of the present invention may comprise the steps of: acquiring odor monitoring information sensed by a plurality of odor sensors; starting to collect odor in an odor collection bag on the basis of the odor monitoring information; acquiring a limit message when the remaining capacity of the odor collection bag is less than or equal to a preset value as the odor is collected; and outputting a notification indicating that replacement of the odor correction bag is required on the basis of the limit message.

Also, the step of starting to collect odor comprises the steps of: acquiring abnormal information indicating that concentration of odor sensed by any one of the plurality of odor sensors which sense different types of odor is greater than or equal to a preset value; and starting to collect the odor in the odor collection bag when the abnormal information is acquired.

Also, the method may further comprise the steps of: acquiring label information indicating a type of odor sensed by the odor sensor which outputs the abnormal information among the plurality of odor sensors, a time of collecting the odor, and a wind direction and wind speed at the time of collecting the odor; and printing a label recording the label information in correspondence with the odor collection.

Also, the method may further comprise the steps of: monitoring a temperature and humidity inside an apparatus in which the plurality of odor sensors are arranged; and recording and printing, on the label, history information on the monitored temperature and humidity when the monitored temperature and humidity are out of a preset range.

Also, the method may further comprise the steps of: acquiring frequency information indicating a frequency of outputting the abnormal information by the plurality of odor sensors which sense different types of odor; and according to the frequency information, recoding and printing, on the label, an over frequency sensor indicating an odor sensor which outputs the abnormal information with a frequency of a first value or more and an insufficient frequency sensor indicating an odor sensor which outputs the abnormal information with a frequency of a second value or less.

Also, the plurality of odor sensors may comprise a first odor sensor, a second odor sensor and a third odor sensor, wherein the first odor sensor may output the abnormal information when a first odor is sensed to have a first concentration or more, the second odor sensor may output the abnormal information when a second odor is sensed to have a second concentration or more, and the third odor sensor may output the abnormal information when a third odor is sensed to have a third concentration or more.

Also, the method may further comprise the step of updating the first concentration, the second concentration, and the third concentration on the basis of the history of the past in which the first odor sensor, the second odor sensor, and the third odor sensor output the abnormal signal, wherein a reference concentration indicating at least one of the first concentration, the second concentration, and the third concentration may increase as the frequency of outputting the abnormal signal gets higher, and decrease as the frequency of outputting the abnormal signal gets lower.

The apparatus for collecting odor according to a second aspect of the present invention may comprise: a plurality of odor sensors which acquire odor monitoring information; and a processor which starts to collect odor in an odor collection bag on the basis of the odor monitoring information, acquires a limit message when the remaining capacity of the odor collection bag is less than or equal to a preset value as the odor is collected, and outputs a notification indicating that replacement of the odor collection bag is required on the basis of the limit message.

### Effect of Invention

According to an embodiment of the present invention, the manager may be supported to easily replace the collection bag in a situation where it is difficult to predict when odor is generated.

Also, the situation where the collection bag is not enough because of frequent generation of unexpected odor and thus the odor cannot be collected may be prevented.

Also, the odor monitoring information and weather information are recorded on the label when collecting the odor, thereby supporting the manger to efficiently manage the odor collection bag and odor information included therein.

The effects of the present invention are not limited to the above-mentioned effects, and it should be understood that the effects of the present disclosure include all effects that could be inferred from the configuration of the invention described in the detailed description of the invention or the appended claims.

### BRIEF DESCRIPTION OF DRAWINGS

FIG. 1 is a block diagram illustrating an example of the constitution of an odor monitoring system according to an embodiment;

FIG. 2 is a block diagram illustrating an example of the constitution of an odor collecting apparatus in FIG. 1;

FIG. 3 is a flow chart illustrating an example of a method for collecting odor by the odor collecting apparatus in FIG. 1; and

FIG. 4 is a block diagram illustrating another example of the constitution of the odor monitoring system in FIG. 1.

### DETAILED MEANS FOR CARRYING OUT THE INVENTION

Hereinafter, the present invention will be explained with reference to the accompanying drawings. The present invention, however, may be modified in various different ways, and should not be construed as limited to the embodiments set forth herein. Also, in order to clearly explain the present invention in the drawings, portions that are not related to the present invention are omitted, and like reference numerals are used to refer to like elements throughout the specification.

Throughout the specification, it will be understood that when a portion is referred to as being “connected” to another portion, it can be “directly connected to” the other portion, or “indirectly connected to” the other portion having intervening portions present. Also, when a component “includes” an element, unless there is another opposite description thereto, it should be understood that the component does not exclude another element but may further include another element.

Hereinafter, examples of the present invention will be explained in more detail with reference to the accompanying drawings.

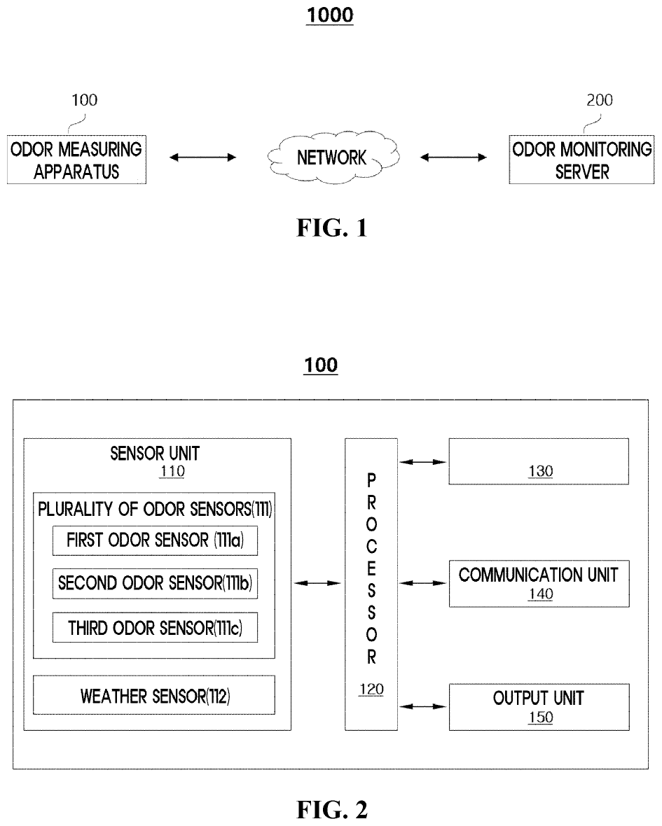

FIG. 1 is a block diagram illustrating an example of the constitution of an odor monitoring system 1000 according to an embodiment.

Referring to FIG. 1, the odor monitoring system 1000 according to an embodiment may comprise an odor collecting apparatus 100 and an odor monitoring server 200 which can communicate with each other through a communications network.

First, the communication network may include various communication networks, such as a local area network (LAN), a metropolitan area network (MAN), a wide area network (WAN), a mobile communication network, etc., regardless of communication aspects such as wired and wireless communications, etc.

The odor collecting apparatus 100 is an apparatus for collecting odor, which may measure odor information, acquire the measured odor information, sense an odor causing substance in real time, and transmit odor information to the odor monitoring server 200 when the odor causing substance is sensed.

In an embodiment, the odor collecting apparatus 100 may be implemented as a mobile apparatus, and for example may be implemented as a form which is portable, simply installed, and movably installed. Additionally, the apparatus 100 may be fixed at a point where it is installed to measure odor information or move from the point installed within a predetermined movement range to measure odor information.

The odor monitoring server 200 may receive odor information acquired from one or more odor collecting apparatus 100 or other constitutional elements, and analyze and manage the odor information generated from a specific point on the basis of the received odor information. Additionally, the odor monitoring server 200 may use the odor information to convert and calculate at least one of the odor type, odor intensity, complex odor, and concentration of odor causing substance.

The odor monitoring server 200 may send an alert message of odor generation to a manager terminal 160 when deciding that odor is generated as a result of analysis of odor information received from the odor collecting apparatus 100.

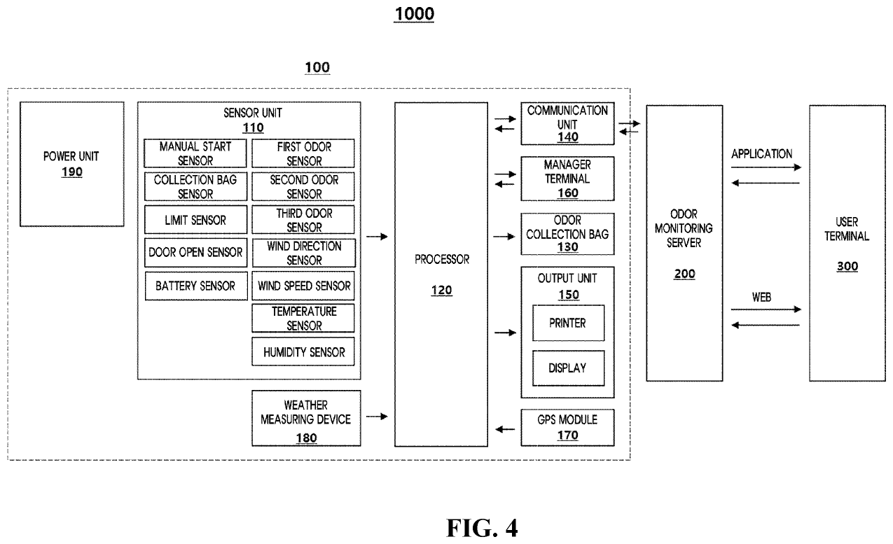

According to an embodiment, the odor monitoring system 1000 may further comprise other common constitutional elements than the constitutional elements illustrated in FIG. 1. The embodiments thereon will be explained below with reference to FIG. 4.

FIG. 2 is a block diagram illustrating an example of the odor collecting apparatus 100 in FIG. 1.

Referring to FIG. 2, the odor collecting apparatus 100 according to an embodiment may comprise a sensor unit 110, an odor collection bag 130, a processor 120, a communication unit 140, and an output unit 150.

The sensor unit 110 may comprise a plurality of odor sensors 111. Each of the plurality of odor sensors 111 may acquire odor monitoring information. According to an embodiment, the odor monitoring information may include the type of odor sensed, the time of collecting odor, and the concentration of odor.

In an embodiment, the plurality of odor sensors 111 may detect whether the concentration of each sensed odor is greater than or equal to a preset value, and output abnormal information indicating that the concentration is greater than or equal to the preset value. For example, when the concentration of each sensed odor is greater than or equal to the preset value identically or differently in each sensor, the plurality of odor sensors 111 may produce abnormal information including the corresponding preset value, the concentration of the sensed odor, the type of odor, the time of collecting odor, and the type of abnormality corresponding thereto to display the same through the output unit 150.

In an embodiment, the plurality of odor sensors 111 may be implemented as a gas sensor which can sense odor causing substances of low concentration. Additionally, the plurality of odor sensors 111 may operate according to smell sensing algorithm implemented similarly to mechanism that a human being senses smell, or may be set to have sensibility corresponding to a sensing level that a human being senses smell, thereby sensing odor causing substances with sensibility similar to that of the human being smells in the air.

In an embodiment, the plurality of odor sensors 111 may comprise at least one of a first odor sensor 111a, a second odor sensor 111b, and a third odor sensor 111c that sense different types of odor.

The first odor sensor 111a may sense a predetermined first odor (i.e., hydrogen sulfide), and for example, may be implemented as a sensor for measuring hydrogen sulfide which can measure a concentration of sulfur-based contaminant including hydrogen sulfide (H₂S) by the sensing for a predetermined first time (i.e., 3 minutes).

In an embodiment, when the first odor is sensed to be greater than or equal to the predetermined first concentration (i.e., 0.5 ppm), the first odor sensor 111a may output abnormal information indicating that the concentration of the sensed first odor is greater than or equal to the preset value.

The second odor sensor 111b may sense a predetermined second odor (i.e., ammonia), and for example, may be implemented as a sensor for measuring ammonia which can measure a concentration of amine-based contaminant including ammonia (NH₃) by the sensing for a predetermined second time (i.e., 3 minutes).

In an embodiment, when the second odor is sensed to be greater than or equal to the predetermined second concentration (i.e., 2 ppm), the second odor sensor 111b may output abnormal information indicating that the concentration of the sensed second odor is greater than or equal to the preset value.

The third odor sensor 111c may sense a predetermined third odor (i.e., total volatile organic compounds (TVOC)), and for example, may be implemented as a sensor for measuring total volatile organic compounds which can measure a concentration of volatile organic compound-based contaminant including TVOC by the sensing for a predetermined third time (i.e., 1 minute).

In an embodiment, when the third odor is sensed to be greater than or equal to the predetermined third concentration (i.e., 0.05 ppm), the third odor sensor 111c may output abnormal information indicating that the concentration of the sensed third odor is greater than or equal to the preset value.

In an embodiment, the plurality of odor sensors 111 may be implemented by at least one of a PID sensor, an electrochemical sensor, and a semiconductor sensor.

In an embodiment, the sensor unit 110 may further comprise a weather sensor 112 which measures weather information, and for example, may sense weather information including at least one of the wind speed, wind direction, temperature and humidity.

The processor 120 may start to collect odor in the odor collection bag 130 on the basis of odor monitoring information sensed by the plurality of odor sensors 111. In an embodiment, the processor 120 may acquire abnormal information indicating that the concentration of odor sensed by any one of the plurality of odor sensors 111 that sense different types of odor is greater than or equal to the preset value, and start to collect odor in the odor collection bag 130 when the abnormal information is acquired.

For example, the processor 120 may control a pressure of the odor collection bag 130 before collecting odor to maintain the bag to be in a predetermined vacuum level. When the above-mentioned abnormal information is acquired, among the plurality (i.e., ten) of odor collection bags 130, any one with no odor collected may be determined. A preparation operation for collecting odor may be performed by doing a cleaning with a predetermined number (i.e., once) using an air blank sample pre-included inside the determined any one odor collection bag 130. When the cleaning is finished, a suction pump may be driven or the pressure of the corresponding odor collection bag 130 may be controlled to collect the odor in the air in the corresponding odor collection bag 130 through a tube.

When the remaining capacity of the odor collection bag 130 is less than or equal to the preset value as the odor is collected, a limit message may be acquired. For example, the limit message including the entire capacity, the remaining capacity, and the ratio of the remaining capacity to the entire capacity may be generated.

In an embodiment, when each of the plurality of odor collection bags 130 is implemented as a disposable collection bag, the processor 120 may receive whether the corresponding collection bag is used from each of the plurality of odor collection bags 130 to generate a limit message according to the remaining capacity (i.e., five) indicating the rest except for the number of already-used odor collection bags in the entire capacity (i.e., ten) indicating the total number of collectable odor collection bags. In another embodiment, when each of the plurality of odor collection bags 130 is implemented as a multi-use collection bag, the processor 120 may receive whether the corresponding collection bag is used from each of the plurality of odor collection bags 130 to generate a limit message according to the remaining capacity (i.e., 10L) indicating the rest except for the already-used collection capacity in the collectable entire capacity (i.e., 50L).

The processor 120 may output a notification indicating that replacement of the odor collection bag 130 is required on the basis of the limit message. For example, when the ratio of the remaining capacity to the entire capacity included in the limit message is less than or equal to the preset value, the processor 120 may generate a notification message including identification information (i.e., numbers 1 to 7) of the odor collection bag 130 required to be replaced, the entire capacity, the remaining capacity, the ratio of the remaining capacity to the entire capacity, and a request message requesting the replacement of the odor collection bag 130, display the notification message through the output unit 150, and transmit the same to the manager terminal 160.

Accordingly, in the situation where it is difficult to predict when odor is generated, the manager may be supported to easily replace the collection bag to prevent the situation where the odor cannot be collected because the collection bag is not enough because of frequent generation of unexpected odor.

In an embodiment, the processor 120 may acquire label information indicating the type of odor sensed by the odor sensor which outputs abnormal information among the plurality of odor sensors 111, the time of collecting odor, and the wind direction and wind speed at the time of collecting odor, and print a label recording the label information in correspondence with the odor collection.

For example, the processor 120 may collect in real time weather information including the wind speed, wind direction, temperature and humidity sensed by the weather sensor 112, and when any one of the plurality of odor sensors 111 outputs the abnormal information, read information on the wind speed, wind direction, temperature and humidity corresponding to the time when the corresponding odor sensor 111 collects odor among the collected weather information, generate label information including the type of odor, the concentration of odor, identification information (i.e., odor collection bag number) of the odor collection bag with odor collected, the time of collecting odor, and the wind speed, wind direction, temperature and humidity at the corresponding time, and print and output the label information on the label through the output unit 150 implemented by including a print module.

Accordingly, the manager may attach the printed label to the odor collection bag 130 in which the corresponding odor is collected to conveniently manage the odor collection bag 130 with odor collected, and odor information and weather information included therein.

In an embodiment, the processor 120 may control the label recording the label information corresponding to the odor collection to be attached to the odor collection bag 130 in which the collection is finished.

In an embodiment, the processor 120 may monitor the temperature and humidity inside an apparatus in which the plurality of odor sensors 111 are arranged. For example, the processor 120 may analyze weather information including the wind speed, wind direction, temperature and humidity sensed by the weather sensor 112 to perform the monitoring for a predetermined time.

In an embodiment, when the monitored temperature and humidity are out of a predetermined range, the processor 120 may record and print the history information on the monitored temperature and humidity on the label. Additionally, when the monitored wind speed and wind direction are out of a predetermined range, the processor 120 may record and print the history information on the monitored wind speed and wind direction on the label.

In an embodiment, the processor 120 may acquire frequency information indicating the frequency that the plurality of odor sensors 111 which sense different types of odor output abnormal information. Here, the frequency information may include the number of outputs of abnormal information for each odor sensor per predetermined unit time.

In an embodiment, the processor 120 may analyze frequency information of each of the plurality of odor sensors 111 to determine each of the plurality of odor sensors 111 as an over frequency sensor or insufficient frequency sensor. Here, the over frequency sensor indicates the odor sensor outputting abnormal information with a frequency of preset first value or more, and the insufficient frequency sensor indicates the odor sensor outputting abnormal information with a frequency of preset second value or less.

For example, the processor 120 may monitor frequency information of each of the plurality of odor sensors 111 for the predetermined time to determine the odor sensor 111 as the over frequency sensor when the frequency information is greater than a preset first reference frequency, and determine the odor sensor 111 as the insufficient frequency sensor when the frequency information is less than a preset second reference frequency. Additionally, according to the update of frequency information, the determination as the over frequency sensor or insufficient frequency sensor may be update.

In an embodiment, the processor 120 may record and print the over frequency sensor and insufficient frequency sensor according to the frequency information on the label. For example, when the first odor sensor 111a determined as the over frequency sensor through the most recent monitoring outputs abnormal information, the processor 120 may include the notification information indicating that the sensor corresponds to the over frequency sensor and frequency information in the label information and print the same on the label.

Accordingly, a user may be notified of information on whether the sensor outputs abnormal information too often or outputs abnormal information too rarely, and may be intuitively notified as to whether the corresponding odor substance is frequently generated or rarely generated in the corresponding area.

Also, the processor 120 may support the user to check whether the sensor determined as the over frequency sensor operates too sensitively or whether the sensor determined as the insufficient frequency sensor operates too insensitively.

Furthermore, in the case of the insufficient frequency sensor, the user may be notified of information that the sensor needs to be replaced into another odor sensor that senses different types of odor, since the type of odor monitored by the insufficient frequency sensor is rarely generated in the corresponding area.

In an embodiment, the processor 120 may update the above-mentioned first concentration, second concentration and third concentration on the basis of the history of the past in which the first odor sensor 111a, second odor sensor 111b and third odor sensor 111c output the abnormal signal. For example, the processor 120 may store and manage the history of the past as to the abnormal signal output by each of the plurality of odor sensors 111, and update the first concentration, second concentration, and third concentration in proportional to the number of outputs of abnormal signal for the predetermined time.

In an embodiment, the processor 120 may increase the reference concentration indicating at least one of the first concentration, second concentration and third concentration as the frequency of outputting the abnormal signal gets higher and reduce the reference concentration as the frequency of outputting the abnormal signal gets lower. In other words, the processor 120 may reduce the number of collections of odor that is collected too often, and increase the number of collections of odor that is collected too rarely.

Accordingly, in the case of odor that is generated frequently in the corresponding area, the reference concentration for determining whether to output the abnormal information is increased, thereby increasing the concentration criteria of odor that determines the abnormality. In the case of odor that is rarely generated in the corresponding area, the reference concentration for determining whether to output the abnormal information is reduced, thereby controlling to determine whether to output the abnormal information flexibly according to the situation in each area.

In an embodiment, the processor 120 may determine the first concentration, second concentration and third concentration by using equation 1 below according to a predetermined period. For example, each odor sensor 111 may update the first concentration, second concentration and third concentration to relatively greater values as the frequency that each odor sensor 111 outputs the abnormal signal gets higher, as the wind speed sensed by the weather sensor 112 gets lower, and as the remaining capacity of the odor collection bag 130 gets lower. Accordingly, the reference concentration for outputting the abnormal information may be tightly updated.

C₁=f₁*(w₀/w)*(v₀/v)*C₀₁

C₂=f₂*(w₀/w)*(v₀/v)*C₀₂

C₃=f₃*(w₀/w)*(v₀/v)*C₀₃ [Equation 1]

(wherein C₁, C₂ and C₃ are the first concentration, second concentration and third concentration, respectively, f₁, f₂ and f₃ are the number of outputs of the abnormal signal by the first odor sensor 111a, second odor sensor 111b and third odor sensor 111c, respectively, for the predetermined time, w is the wind speed sensed by the weather sensor 112, w₀ is the reference wind speed which can be set by the user, v is the remaining capacity of the odor collection bag 130, v₀ is the reference remaining capacity which can be set by the user, and C₀₁, C₀₂, and C₀₃ are the first default concentration, second default concentration and third default concentration which can be set by the user, respectively).

In an embodiment, when the frequency of outputting the abnormal signal by the first odor sensor 111a is still less than or equal to the preset value even though the first concentration is updated to be reduced to be greater than or equal to the predetermined number, the processor 120 may output a message indicating that the first odor sensor 111a is unnecessary. In other words, the information that the type of odor that is collected too little needs not to be sensed may be provided.

In an embodiment, when the frequency of outputting the abnormal signal by the first odor sensor 111a is still greater than or equal to the preset value even though the first concentration is updated to be increased to be greater than or equal to the predetermined number, the processor 120 may output a message indicating that the type of odor sensed by the first odor sensor 111a needs not to be sensed since said odor is common. In other words, the information that the type of odor that is collected too much is the common odor and thus needs not to be sensed may be provided.

In an embodiment, when the frequency included in a specific time zone (i.e., 9 am to 12 pm) in which the first odor sensor 111a outputs the abnormal signal is greater than or equal to the preset value, the processor 120 may output a message indicating that the sensing is necessary, regardless of the frequency that the first odor sensor outputs the abnormal signal, since the type of odor sensed by the first odor sensor 112 is common odor with tendency. In other words, even though the type of odor is collected too much, if there is a tendency in the time zone in which the odor is collected, the monitoring is necessary, and thus the processor 120 may output a message indicating that the sensing is needed.

Likewise, the processor 120 may perform the same operation as above for the second odor sensor 111b or third odor sensor 111c.

In an embodiment, when the odor sensor 111 determined as the over frequency sensor outputs the abnormal information, the processor 120 may collect odor according to the predetermined first time (i.e., 1 minute) corresponding to instant collection when collecting odor. Additionally, when the odor sensor 111 determined as the insufficient frequency sensor outputs the abnormal information, the processor 120 may collect odor according to the predetermined second time (greater than the first time) (i.e., 5 minutes) corresponding to continuous collection when collecting odor.

Accordingly, based on the real-time data, the collection time for collecting the odor that instantaneously appears and disappears and for collecting the odor that continuously lasts may be differently adjusted.

In an embodiment, the processor 120 may predict a moving path of odor generation on the basis of weather information which includes the wind speed, wind direction, temperature and humidity sensed by the weather sensor 112. For example, the processor 120 may analyze the type of odor sensed by the odor sensor which outputs the abnormal information, the concentration of odor, the time of collecting odor, and the wind direction, wind speed, temperature and humidity at the time of collecting odor. Additionally, the processor 120 may output moving path prediction information by generating prediction information on at least one of the moving direction of odor according to the time on the basis of location information where the odor collecting apparatus 100 is arranged, the concentration for each movement section, and the point where odor is generated on the basis of neighboring facilities or regional information, and mapping the prediction information on a pre-stored map.

In an embodiment, the processor 120 may calculate reliability for the prediction of the moving path on the basis of the wind speed, wind direction, temperature and humidity sensed, and output the same with the moving path prediction information.

In an embodiment, the processor 120 may analyze the pattern of odor generation on the basis of the history of the weather information acquired from the weather sensor 112 and the odor information acquired from the plurality of odor sensors 111, and predict odor generation from the pattern of odor generation. For example, the processor 120 may analyze the pattern of odor generation as to the time zone when the abnormal information on the odor is mainly output, weather information, location information, facilities information included in the location information, etc., to calculate the possibility of odor generation for each time zone and each type of odor. When the calculated possibility of odor generation is greater than the preset value, the prediction information including a result of the corresponding analysis may be transmitted to or displayed on a manager terminal (not illustrated), thereby outputting a notification message requesting the manager to be cautious about odor generation.

In an embodiment, when the odor is collected, the processor 120 may transmit a notification message including at least one of the type of odor, the concentration of odor, the time of collecting odor, and the wind direction, wind speed, temperature and humidity at the time of collecting odor to the manager terminal 160 which corresponds to the predetermined manager, and may provide the information on other operations mentioned above on websites or applications through an application installed in the manager terminal 160 or user terminal 300.

The processor 120 according to an embodiment may be implemented as a central processor unit (CPU) that controls the overall operation of the odor collecting apparatus 100, and may be electrically connected to the communication unit 110 and storage unit 130 to control data flow therebetween.

The odor collection bag 130 may collect odor according to the control of the processor 120. In an embodiment, the odor collection bag 130 may perform pre-programmed sequential control and pressure control on the basis of the control of the processor 120, and accordingly, may maintain the bag to be in the vacuum state, clean the collection bag by using the air blank sample, or start to collect the sample (i.e., odor collection).

In an embodiment, a plurality of odor collection bags 130 may be provided, and at least one of whether each of the bags collects odor, the entire capacity, collection capacity, and remaining capacity may be provided to the processor 120.

In an embodiment, the odor collection bag 130 may include at least one of a vacuum control module which performs vacuum control, a plurality of collection bags which collect samples, a pressure sensor which senses the inner pressure, a pipe through which the sample moves, a valve which collects the sample, and a suction pump which pumps odor. In an embodiment, the pipe and valve may be implemented using poly tetra fluoro ethylene (PTFE).

The communication unit 140 according to an embodiment may communicate with other devices (i.e., terminals, servers) through a communications network. For example, the communication unit 140 may include all types of wired and wireless communication apparatuses which can communicate with other devices, and transmit the above-mentioned information to the odor monitoring server 200.

The output unit 150 according to an embodiment may include at least one of a display module for visualizing the output information and a print module for printing the output information. For example, the output unit 150, as mentioned above, may visualize, through the display module implemented as a touch panel, the odor monitoring information on the sensed hydrogen sulfide, ammonia, TVOCs, etc., the weather information on temperature, humidity, wind direction, wind speed, etc., and the information on odor type, odor intensity and complex odor (i.e., dilution factor) determined based on the above.

Additionally, for example, the output unit 150 may print, through the printer module implemented as a label printer, the label information including the odor monitoring information and weather information on the label implemented as a sticker paper.

Accordingly, the output unit 150 may print the weather situation when collecting the sample and use a sticker-type paper to support the sticker in which the place of colleting the sample, the collecting time, the odor type, the odor concentration, the weather information, etc. are printed to be attached to the bag in which the sample is collected.

Also, it could be understood by a person having ordinary knowledge in the related technical field that other common constitutional elements than the constitutional elements illustrated in FIG. 2 may be further included in the odor collecting apparatus 100. For example, the odor collecting apparatus 100 may further include a memory for storing data required throughout the operation of collecting odor, an interface for receiving a user input, etc. Also, according to another embodiment, a part of the constitutional elements illustrated in FIG. 2 may be omitted.

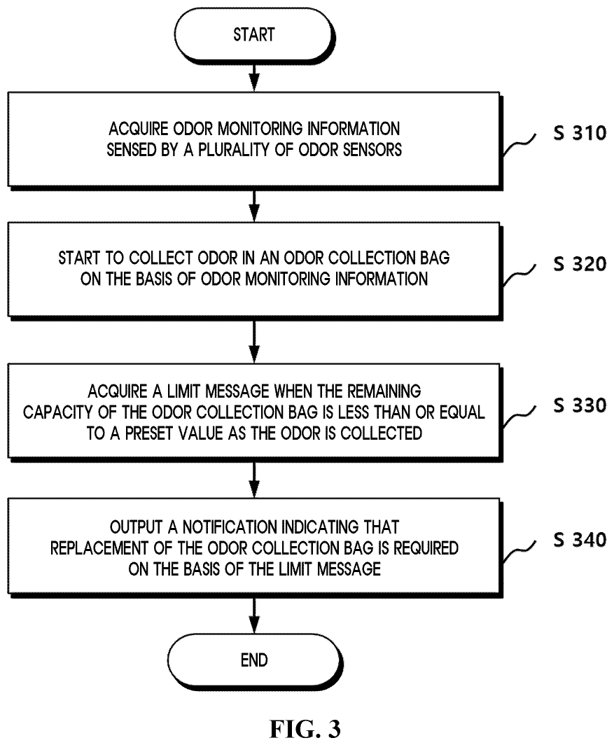

FIG. 3 is a flow chart illustrating an example of a method for collecting odor by the odor collecting apparatus in FIG. 1.

In step S310, the odor collecting apparatus 100 according to an embodiment may acquire odor monitoring information sensed by a plurality of odor sensors 111. In an embodiment, the plurality of odor sensors 111 may sense different types of odor, and include a first odor sensor 111a which senses a first odor (i.e., hydrogen sulfide), a second odor sensor 111b which senses a second odor (i.e., ammonia), and a third sensor 111c which senses a third odor (i.e., total volatile organic compounds (TVOC)).

In step S320, the odor collecting apparatus 100 according to an embodiment may start to collect odor in an odor collection bag on the basis of the odor monitoring information. In an embodiment, the odor collecting apparatus 100 may acquire abnormal information indicating that the concentration of odor sensed by any one of the plurality of odor sensors 111 that sense different types of odor is greater than or equal to the preset value, and start to collect the odor in the odor collection bag 130 when the abnormal information is acquired.

In step S330, the odor collecting apparatus 100 according to an embodiment may acquire a limit message when the remaining capacity of the odor collection bag 130 is less than or equal to the preset value as the odor is collected. In an embodiment, the odor collecting apparatus 100 may acquire label information indicating the type of odor sensed by the odor sensor that outputs the abnormal information among the plurality of odor sensors 111, the time of collecting odor, and the wind direction and wind speed at the time of collecting odor.

In step S340, the odor collecting apparatus 100 according to an embodiment may output a notification indicating that replacement of the odor collection bag is required on the basis of the limit message. In an embodiment, the odor collecting apparatus 100 may print the label recording the label information in correspondence with the odor collection.

According to an embodiment of the present invention, by notifying that replacement of the odor collection bag 130 is required based on the remaining capacity of the odor collection bag 130 when collecting odor, the manger may be supported to easily replace the collection bag in a situation where it is difficult to predict when the odor is generated, and also the situation where the collection bag is not enough because of frequent generation of unexpected odor and thus the odor cannot be collected may be prevented.

Also, by recording the odor monitoring information and weather information on the label when collecting odor, the manager may be supported to efficiently manage the odor collection bag 130 and odor information included therein.

FIG. 4 is a block diagram illustrating another example of the constitution of the odor monitoring system 1000 in FIG. 1.

Referring to FIG. 4, the odor monitoring system 1000 according to an embodiment may comprise an odor collecting apparatus 100, an odor monitoring server 200 and a user terminal 300 which can communicate with each other through a communications network.

In an embodiment, a sensor unit 110 included in the odor collecting apparatus 100 may include a first odor sensor 111a which senses a first odor, a second odor sensor 111b which senses a second odor, a third odor sensor 111c which senses a third odor, a wind direction sensor which senses the wind direction, a wind speed sensor which senses the wind speed, a temperature sensor which senses the temperature, a humidity sensor which senses the humidity, a manual start sensor which senses whether the manual start is made (i.e., odor collection), a collection bag sensor which senses whether the odor collection bag 130 is replaced, a limit sensor which senses the remaining capacity of each odor collection bag 130 to acquire a limit message, a door open sensor which senses whether the door of the odor collecting apparatus 100 or odor collection bag 130 is open or closed, and a battery sensor which senses the state of a battery (i.e., remaining capacity).

In an embodiment, an output unit 150 included in the odor collecting apparatus 100 may include a printer implemented as a label printer and a display implemented as LCD.

In an embodiment, the odor collecting apparatus 100 may further include a manager terminal 160 which can be used as a terminal at the site. For example, the odor collecting apparatus 100 may be electrically connected or connected through a local area communication module, to transmit the information including the type of sensed odor, the concentration of odor, and the time of collecting the odor when the odor is collected as a notification message to the manager terminal 160.

In an embodiment, the odor collecting apparatus 100 may further include a GPS module 170, acquire the location information of a place where the odor collecting apparatus 100 is installed using the GPS module 170, perform the odor analysis based thereon, and transmit the monitoring information to the odor monitoring server 200.

In an embodiment, the odor collecting apparatus 100 may further include a weather measuring device 180 which includes a weather sensor 112 implemented independently from the sensor unit 110. Accordingly, the temperature and humidity inside the apparatus in which the sensor unit 110 is arranged are received from the weather measuring device 180 of the odor collecting apparatus 100 to perform the monitoring based thereon.

In an embodiment, the odor collecting apparatus 100 may further include a power unit 190, and the power unit 190 may include a solar cells panel which acquires electrical energy using sunlight, and a battery module which stores energy acquired through the solar cells panel.

In an embodiment, the odor monitoring server 200 may receive odor information from the odor collecting apparatus 100 to store and monitor the same, and process the stored monitoring information to provide the same to a user through an application installed on the user terminal 300 or on web sites.

In an embodiment, the user terminal 300 may install an application for odor information providing services provided by the odor monitoring server 200 and store the application in a memory. For example, the user terminal 300 may be implemented as a cellular phone, a smart phone, a personal digital assistant (PDA), a portable multimedia player (PMP), a tablet PC, etc., and communicate with the odor monitoring server 200 through the installed application to receive information on odor measured by the odor collecting apparatus 100.

According to an embodiment of the present invention, when the odor whose concentration is greater than or equal to a reference concentration is generated, the odor collecting apparatus 100 may automatically collect the odor, print the weather information at the time of collecting the odor on the label, and attach the same to the collection bag, thereby supporting easy management of the collection bag.

Additionally, when the odor is collected, the information may be transmitted to managers or designators using a text message, and the result of real-time odor monitoring may be stored in the server so that the odor collection may be precisely performed.

Meanwhile, the above-described method can be written as a program that can be executed in a computer, it can be implemented in a general-purpose digital computer to operate the program using a computer-readable recording medium. In addition, the structure of the data used in the above-described method can be recorded on the computer-readable recording medium through various means. The computer-readable recording medium may include a storage medium such as a magnetic storage medium (for example, a ROM, a RAM, a USB, a floppy disk, a hard disk, etc.) and an optical reading medium (for example, a CD-ROM, a DVD, etc.).

The above-described description of the present invention is intended for illustration, and a person having ordinary knowledge in the art to which the present invention pertains will understand that the present invention may be easily modified in other specific forms without changing the technical spirit or essential features of the present invention. Therefore, it should be understood that the embodiments described above are exemplary in all respects and not restrictive. For example, each component described as a single type may be implemented in a distributed manner, and similarly, components described as distributed may be implemented in a combined form.

The scope of the present invention is defined by the accompanying claims. It should be construed that all modifications and embodiments derived from the meaning and scope of the claims and their equivalents fall within the scope of the present invention.

1. A method for collecting odor, comprising the steps of:
acquiring odor monitoring information sensed by a plurality of odor sensors;
starting to collect odor in an odor collection bag on the basis of the odor monitoring information;
acquiring a limit message when the remaining capacity of the odor collection bag is less than or equal to a preset value as the odor is collected; and
outputting a notification indicating that replacement of the odor correction bag is required on the basis of the limit message.

2. The method of claim 1, wherein the step of starting to collect odor comprises the steps of:
acquiring abnormal information indicating that concentration of odor sensed by any one of the plurality of odor sensors which sense different types of odor is greater than or equal to a preset value; and
starting to collect the odor in the odor collection bag when the abnormal information is acquired.

3. The method of claim 2, further comprising the steps of:
acquiring label information indicating a type of odor sensed by the odor sensor which outputs the abnormal information among the plurality of odor sensors, a time of collecting the odor, and a wind direction and wind speed at the time of collecting the odor; and
printing a label recording the label information in correspondence with the odor collection.

4. The method of claim 3, further comprising the steps of:
monitoring a temperature and humidity inside an apparatus in which the plurality of odor sensors are arranged; and
recording and printing, on a label, history information on the monitored temperature and humidity when the monitored temperature and humidity are out of a preset range.

5. The method of claim 3, further comprising the steps of:
acquiring frequency information indicating a frequency of outputting the abnormal information by the plurality of odor sensors which sense different types of odor; and
according to the frequency information, recoding and printing, on a label, an over frequency sensor indicating an odor sensor which outputs the abnormal information with a frequency of a first value or more and an insufficient frequency sensor indicating an odor sensor which outputs the abnormal information with a frequency of a second value or less.

6. The method of claim 2, wherein the plurality of odor sensors comprises a first odor sensor, a second odor sensor and a third odor sensor,
wherein the first odor sensor outputs the abnormal information when a first odor is sensed to have a first concentration or more,
the second odor sensor outputs the abnormal information when a second odor is sensed to have a second concentration or more, and
the third odor sensor outputs the abnormal information when a third odor is sensed to have a third concentration or more.

7. An apparatus for collecting odor, comprising:
a plurality of odor sensors which acquire odor monitoring information; and
a processor which starts to collect odor in an odor collection bag on the basis of the odor monitoring information, acquires a limit message when the remaining capacity of the odor collection bag is less than or equal to a preset value as the odor is collected, and outputs a notification indicating that replacement of the odor collection bag is required on the basis of the limit message.
